(19) United States
(12) Patent Application Publication
(10) Pub. No.: US 2026/0038663 A1
(43) Pub. Date: Feb. 5, 2026

(54) SELECTIVELY ACCESSIBLE CONTAINERS FOR DISPENSING OF MEDICAMENT VIALS

(71) Applicant: CareFusion 303, Inc., San Diego, CA (US)

(72) Inventors: Daniel M. Abal, San Diego, CA (US); David Hoard, Escondido, CA (US); Colburn Leonard Jones, Butler, PA (US)

(21) Appl. No.: 19/277,792

(22) Filed: Jul. 23, 2025

Related U.S. Application Data
(60) Provisional application No. 63/677,484, filed on Jul. 31, 2024.

(51) Int. Cl. G16H 20/13; A61J 7/00; G16H 40/20

(52) U.S. Cl. CPC G16H 20/13; A61J 7/0076; G16H 40/20

(57) ABSTRACT

A container for a machine for dispensing vials containing a medicament includes: a housing including a receptacle with an internal compartment and a lid, the receptacle including a track in the internal compartment, the track configured to receive a plurality of medicament vials; a curtain connected with the housing, the curtain configured to form a gap above the track sized to permit access to a vial stored in the track; a drive mechanism mounted to the housing; and a controller operatively connected with the drive mechanism. The drive mechanism is configured to receive signals from the controller to selectively drive either the curtain or vials in the track so that a single vial is positioned below the gap.

### CROSS-REFERENCE TO RELATED APPLICATION

The present application claims priority to U.S. Provisional Patent Application No. 63/677,484, filed on Jul. 31, 2024, the disclosure of which is incorporated herein by reference in its entirety.

### FIELD

The present disclosure generally relates to systems and methods of automatically dispensing pharmaceuticals.

### BACKGROUND

Hospitals and other health care setting (e.g., skilled nursing facilities, outpatient facilities, retirement homes and communities, etc.) have a need to provide secure storage for certain medications such as narcotics and controlled substances while still making the medications available to care givers. One method of accomplishing this is to use lidded containers where the lid can only be opened when the container is connected to a power source and processor that can send the appropriate digital commands to permit opening. The lidded container is filled with a medication in the pharmacy and the lid closed.

In many instances, such containers are stored in a local automated dispensing machine (ADM). In many instances the ADM includes one or more drawers that slide between open and closed positions to provide access thereto. The secure containers are mounted within the drawers. The ADM can provide power and can send the commands to open the lid of the container, and can also regulate the opening of the drawers. Software of the ADM is configured to open the drawer and/or the lid of the container only after certain requirements are met, such as verification that the individual accessing the container is authorized to do so.

Selectively accessible containers have taken a number of different forms. Exemplary configurations are shown in U.S. Pat. Nos. 9,687,075; 10,029,829 and 10,435,918, each of which is hereby incorporated by referenced herein in its entirety. Typically medications are stored in a vial, jar, bottle, or the like that is stored within the container, or in other instances the medications are loosely stored within the container. In either instance the medications are secure, as access is prohibited to those without appropriate clearance/permission (i.e., the lid of the container will not open without such clearance/permission).

Although ADMs with secure containers may be adequate for many medications, there are some regulated medications that have additional levels of security. In addition, some medications are provided in packaging that differs from the vials and bottles discussed above. As such, it may be desirable to provide additional configurations for secure dispensing of medications in hospital settings.

### SUMMARY

As a first aspect, embodiments of the invention are directed to a container for a machine for dispensing vials containing a medicament. The container comprises: a housing including a receptacle with an internal compartment and a lid, the receptacle including a track in the internal compartment, the track configured to receive a plurality of medicament vials; a curtain connected with the housing, the curtain configured to form a gap above the track sized to permit access to a vial stored in the track; a drive mechanism mounted to the housing; and a controller operatively connected with the drive mechanism. The drive mechanism is configured to receive signals from the controller to selectively drive either the curtain or vials in the track so that a single vial is positioned below the gap.

As a second aspect, embodiments of the invention are directed to a container for a machine for dispensing vials containing a medicament comprising: a housing including a receptacle with an internal compartment and a lid, the receptacle including a track in the internal compartment, the track configured to receive a plurality of medicament vials; a curtain connected with the housing, the curtain configured to form a gap above the track sized to permit access to a vial stored in the track; a drive mechanism mounted to the housing and to the curtain; and a controller operatively connected with the drive mechanism. The drive mechanism is configured to receive signals from the controller to selectively drive the curtain relative to the housing so that a single vial is positioned below the gap.

As a third aspect, embodiments of the invention are directed to a container for a machine for dispensing vials containing a medicament comprising: a housing including a receptacle with an internal compartment and a lid, the receptacle including a track in the internal compartment, the track configured to receive a plurality of medicament vials; a curtain connected with the housing, the curtain configured to form a gap above the track sized to permit access to a vial stored in the track; a drive mechanism mounted to the housing; and a controller operatively connected with the drive mechanism. The drive mechanism is configured to receive signals from the controller to selectively drive vials in the track so that a single vial is positioned below the gap.

### BRIEF DESCRIPTION OF THE FIGURES

FIG. 1 is a perspective view of a selective access cabinet that includes selectively accessible containers according to embodiments of the invention.

FIG. 2 is a side perspective view of a container for selectively accessing vials according to embodiments of the invention.

FIG. 3 is a perspective view of an exemplary vial to be housed in the container of FIG. 2.

FIG. 4 is a top view of a container according to embodiments of the invention with the lid and curtain removed.

FIG. 5 is a perspective view of a curtain for the container of FIG. 4.

FIG. 6 is a section view of the curtain of FIG. 5

FIG. 7 is an enlarged, partial perspective view of the curtain of FIG. 5 and a side wall of the container of FIG. 4.

FIG. 8 is a top view of the container of FIG. 4 with the curtain in place and permitting access to one vial housed in the container.

FIG. 9 is a side section view of the container as shown in FIG. 8.

FIG. 10 is an enlarged, partial perspective view of a curtain according to alternative embodiments of the invention.

FIG. 11 is a perspective view of the lower portion of a container according to alternative embodiments of the invention.

FIG. 12 is a top view of the container of FIG. 11.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout and different embodiments of like elements can be designated using a different number of superscript indicator apostrophes (e.g., 10′, 10″, 10′″).

In the figures, certain layers, components or features may be exaggerated for clarity, and broken lines illustrate optional features or operations unless specified otherwise. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

It is noted that aspects of the invention described with respect to one embodiment, may be incorporated in a different embodiment although not specifically described relative thereto. That is, all embodiments and/or features of any embodiment can be combined in any way and/or combination. Objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

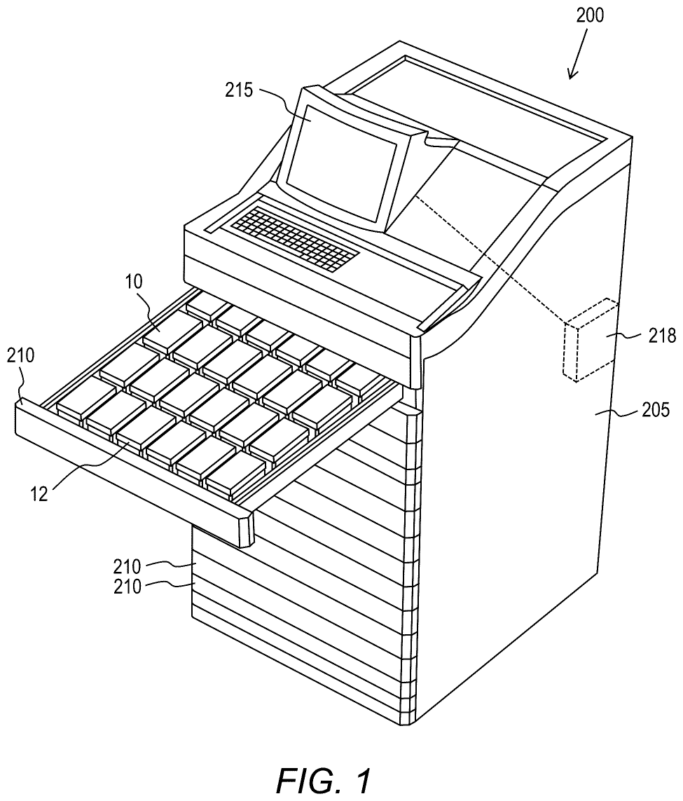

Referring now to the figures. FIG. 1 illustrates an automated dispensing machine (ADM)) for pharmaceuticals, designated broadly at 200, that may be used in hospital and other health care settings. The machine 200 includes a generally box-shaped cabinet 205 with a number of vertically-stacked drawers 210 that are slidable relative to the remainder of the cabinet 205. Each of the drawers 210 is locked in its closed position via a locking mechanism (not shown) that prevents access to the drawer 210 without permission. Either the drawer 210, the locking mechanism, or both are operatively connected with a controller 218 in the machine 200 that prevents the drawer 210 from opening without access having been granted in the manner described below.

Within each drawer 210 are multiple containers 10, each of which contains a bulk supply of a medicament or other medical item (e.g., a bandage, syringe, or the like). The containers 10 may include a vial or bottle containing a bulk supply of pills, tablets, etc., or the containers 10 may include the pills or other items loosely contained within the container. Each container 10 includes a local controller (e.g., a printed circuit board (PCB)) that interfaces with the controller 218. Typically the interface connection between the container 10 and the controller 218 is established when the container 10 is installed in the drawer 210 (e.g., the PCB may include a port or socket that connects with a mating plug in the drawer 210 that is connected to the controller 218). Exemplary interface connections are discussed in, for example, U.S. Pat. No. 9,687,075, supra.

In addition, each of the containers 10 is locked via a locking mechanism (not shown herein) to prevent access to a user (e.g., a medical professional such as a nurse or medical technician) unless access is permitted in the manner described below. Typically, each container 10 is configured such that its lid 12 pivots to an open position to provide access to the interior of the container 10 where the medicaments or other medical items reside. Exemplary locking mechanisms for the drawers 210 and containers 10 are shown, for example, in U.S. Pat. Nos. 10,029,829 and 10,435,918, supra. An exemplary machine 200 with such drawers 210 and containers 10 is the Pyxis™ system, available from Becton, Dickinson & Company (Franklin Lakes, New Jersey),

A user interface 215 (represented herein by a display monitor and a keyboard) is operatively connected with the locking mechanisms of the drawers 210 and containers 10. The user interface 215 is operatively connected with the controller 218 that is, as described above, operatively connected with the drawers 210 and containers 10. The controller 218 is configured to enable a user to selectively open one of the drawers 210 via data input via the user interface 215 (typically related to the identity of the user and a specific patient or group of patients). In addition, the controller 218 is configured to enable the user to selectively open a specific container 10 via inputs into the user interface 215. In some embodiments, the drawers 210 are configured so that the drawer 210 of interest slides open automatically upon the correct input from the technician. Also, in some embodiments, the containers 10 are configured so that the container 10 that contains the medicament or medical item of interest opens automatically once the drawer 210 is opened. In other words, when a user inputs the correct information into the user interface 215 to qualify for permission to access the medicament or medical item, the controller 218 signals the correct drawer 210 and correct container 10 to open (i.e., to unlock their respective locking mechanisms) to provide access to the contents of that container 10 to the user. As this occurs, access to other containers 10 within that drawer 210 is typically denied unless the input provided by the technician also granted permission to more than one container 10.

In some embodiments, the controller 218 will be operatively connected with an external device, such as a personal or mainframe computer, that provides input information regarding prescriptions, patients, medical personnel that may access the containers (e.g., locally or over a network). In other embodiments, the controller 218 may be a stand-alone computer that directly receives manual input from a pharmacist or other operator through the user interface 215. An exemplary controller 218 is a conventional microprocessor-based personal computer. The controller 218 may also be divided into multiple computers, networks, processors, etc., that combine and/or share operations. For example, one portion of the controller 218 may be internal to the machine 200 and control certain operations of the machine 200, and another portion of the controller 218 may be external to the machine 218 and control other operations of the machine 200. Alternatively, the user interface may be or include a handheld device.

The containers 10 mounted in the machine 200 are similar in size and function, and therefore are potentially interchangeable with each other, which enables the machine 200 to be set up as needed (e.g., if one medicament experiences heavy usage, additional containers 10 of that medicament may be included in the machine 200). However, as discussed above, in some instances it may be desirable to supply medicaments that are provided in a different form than the bulk forms discussed above.

For example, in some instances a container 10 may house a vial that is considerably smaller than the interior of the container 10. As such, the container 10 would have sufficient room to house multiple vials in the same container 10. An arrangement that would house multiple vials would free up other containers 10 in the same machine 200 to house additional medicaments. However, for security purposes such a container 10 may require some way of restricting access to only one vial at a time.

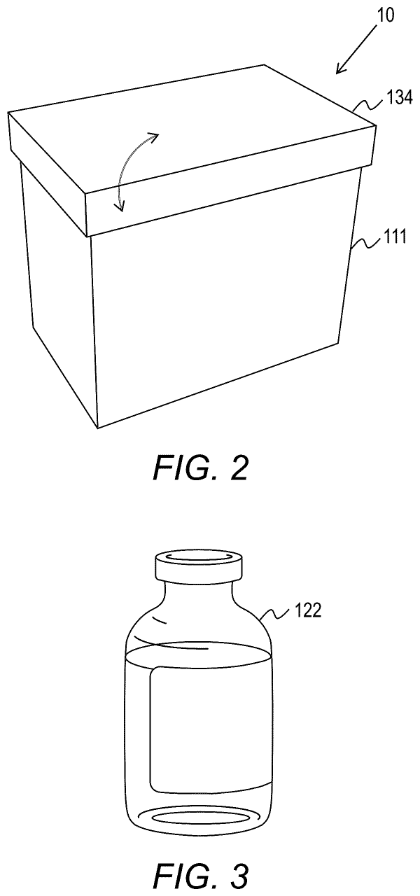

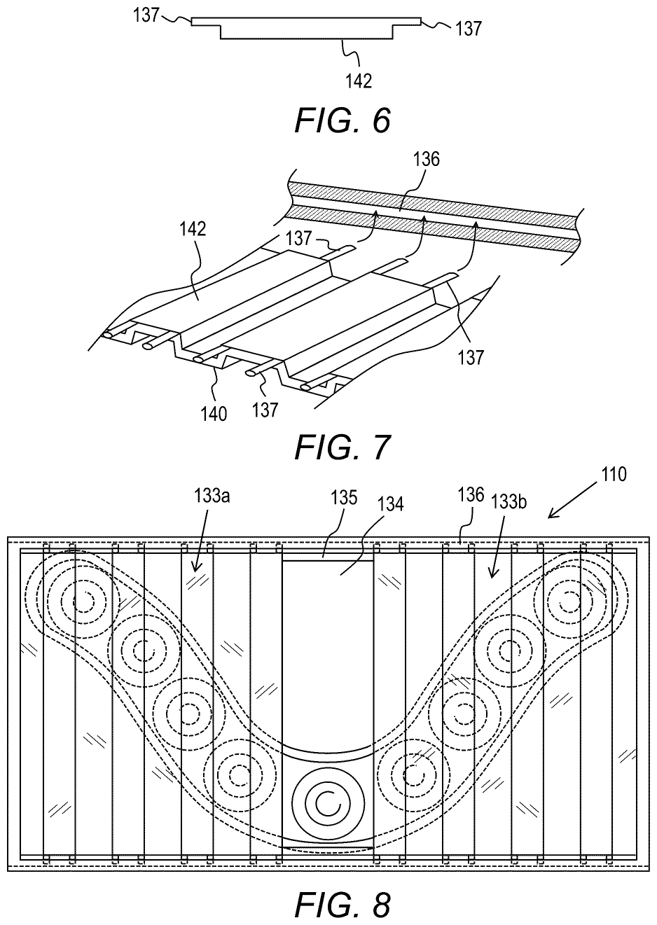

An exemplary container that can house multiple vials is illustrated in FIGS. 2-9 and designated broadly at 110. As seen in FIG. 2, the container 110 is box-shaped like the containers 10, having a receptacle 111 and a lid 134, and is generally of a similar size and shape (i.e., it is similar in height, width and depth) as the containers 10. The lid 134 is lockable (in some embodiments with a mechanism like those employed with containers 10—see, for example, U.S. Pat. Nos. 10,029,829 and 10,435,918, supra.) to prevent access to the receptacle 111 when the drawer 210 is opened to permit access to a different container 10.

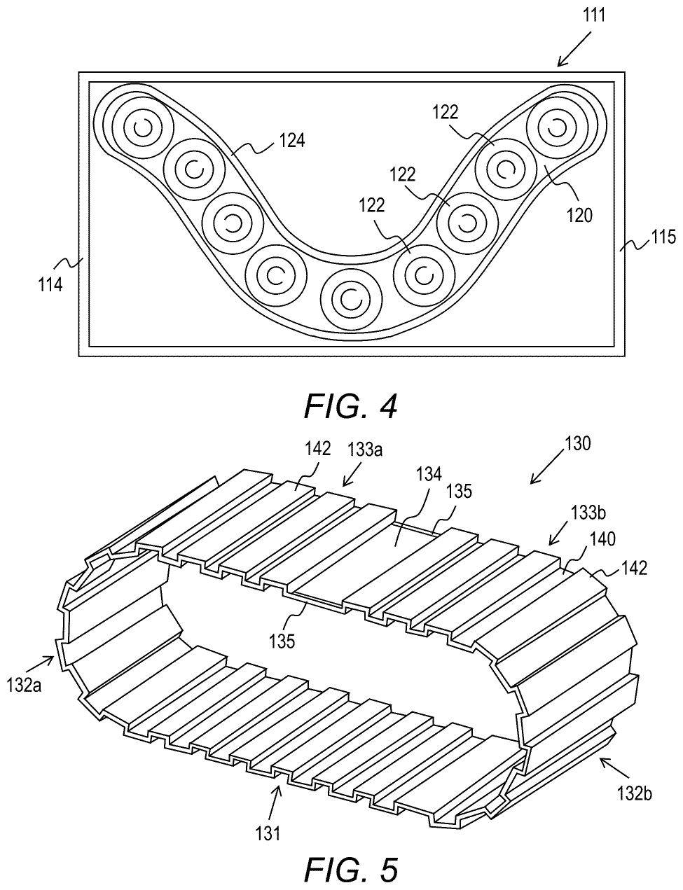

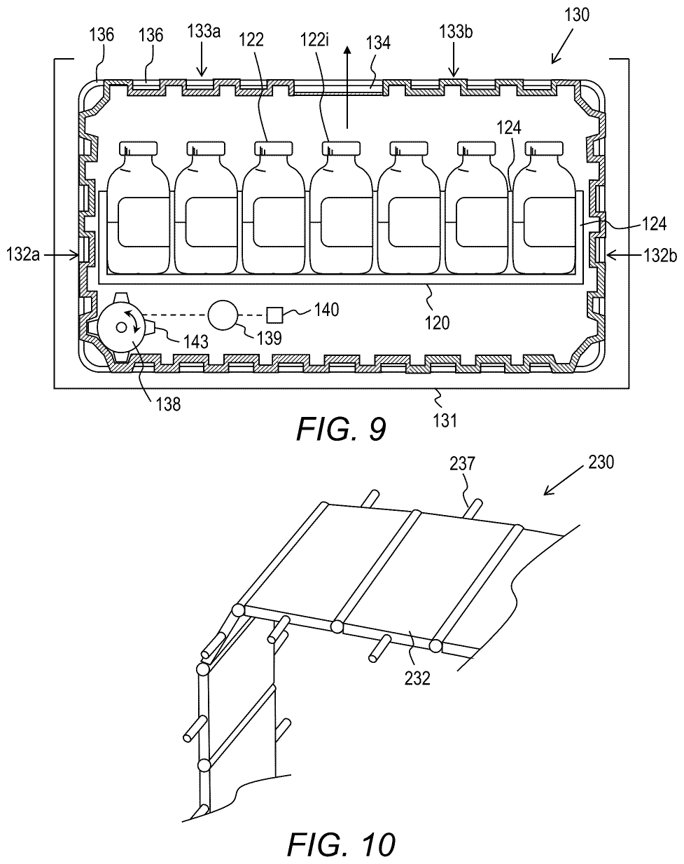

Referring now to FIG. 4, a track 120 is located within the receptacle 111. The track 120 is sized and configured to receive and store a plurality of vials 122 (see FIG. 3), each of which contains the same medicament, or in some embodiments may contain a different medicament. In the illustrated embodiment, the track 120 follows an arcuate path, which enables additional vials 122 to be included within the track 120 and, in turn, within the container 110. However, in other embodiments the track 120 may follow a different path (e.g., straight, zig-zag, serpentine, etc.), any of which may be chosen to increase or maximize the number of vials 122 that can be contained therein. However, in any instance the track 120 should be configured so that it is routed to gradually extend from the front wall 114 to the rear wall 115 without doubling back on itself. The track 120 has side walls 124 (see also FIG. 9) that are of sufficient height that the vials 122 are maintained in an upright orientation and are not prone to tipping over. Also, the track 120 may include indentions (e.g., circular recesses) configured to receive vials 122 and maintain them in place within the track 120. Further, the track 120 or the surrounding environment may include sensors, scanners or the like to confirm the identity of vials 122 positioned in the track 120.

In some embodiments, the track 120 may be removeable, such that it may be replaced by a track 120 having different dimensions (e.g., a different width) that are appropriate for vials of a different size. Providing tracks 120 of multiple sizes may permit the container 110 to be customized to accommodate vials 122 of different sizes as work flow demands.

Referring now to FIGS. 5-9, a movable “curtain” 130 is located within the receptacle 111. The curtain 130 is configured to have a lower horizontal run 131 that underlies the track 120, two vertical runs 132a, 132b that merge at their lower ends with the ends of the horizontal run 131 and extend upwardly therefrom, and two upper horizontal runs 133a, 133b that are attached to the upper ends of the vertical runs 132a, 132b and extend toward each other above the vials 122 located in the track 120. As can be seen in FIGS. 5, 8 and 9, the upper horizontal runs 133a, 133b do not meet; instead, their free ends define a gap 134 above the vials 122 in the track 120. In some embodiments, tethers 135 may extend between the upper horizontal runs 133a, 133b on either side of the gap 134 to maintain the width and depth of the gap 134. It should also be noted that the depth of the gap 134 (i.e., the direction from left to right in FIG. 8) is sufficient that one vial positioned directly below the gap 134 may be removed from the track 120 through the gap 134, but the remaining vials 122 in the container 110 remain inaccessible.

As seen in FIGS. 7 and 9, the side edges of the curtain 130 may be received in tracks or channels 136 located in the side walls of the container 110 that help to define the shape and travel path of the curtain 130. As shown in FIG. 9, the channels 136 may be endless channels. Pins or posts 137 may extend laterally from the side edges of the curtain 130 into the channels 136 to help to maintain the curtain 130 in place. A drive wheel 138 is mounted to engage the inner surface of the curtain 130. The drive wheel 138 is coupled with a drive motor 139 that is operatively connected a controller 140 (typically in the form of a PCB-shown schematically in FIG. 9) that is in turn operatively connected with the controller of the cabinet 200.

The curtain 130 may take any of several forms, any of which permit the curtain 130 to flex as it is moved within the receptacle 111. As one example, and as shown in FIG. 5, the curtain 130 may be a single unitary component, formed for example from a plastic material, wherein periodically a thinned flexible section 140 is interposed between two more rigid sections 142. The result is a curtain 130 with a “corrugated” profile. With such a curtain, the drive wheel 138 may have teeth 143 that are received in the space between the flexible sections 140 (i.e., radially inwardly from the rigid sections 142) that enable the drive wheel 138 to drive the curtain 130 along a path defined by the channels 136.

In operation, the lid 134 of the container 110 is opened, and the track 120 is loaded with vials 122. If the vials 122 contain different medicaments, loading may be accompanied by scanning of the vials 122 to identify the medicaments within the vials 122. Once the vials 122 are loaded, the lid 134 is closed, and the container 110 is then placed in position in the drawer 210 of the machine 200, where it remains securely inaccessible until dispensing.

When a user inputs information into the user interface 215 that requests the dispensing of a blister card 100 from the container 110, the controller 218 first signals the correct drawer 210 to open. The controller 218 then determines whether a specific vial 122i (see FIG. 8) to be dispensed is positioned beneath the gap 134 in the curtain 130. If the vial 122i is already positioned beneath the gap 134 (as is the case in FIG. 8), the curtain 130 need not move, and the user can remove the vial 122i from the container 110. However, if the vial 122i is not positioned beneath the gap 134, the controller 218 signals the drive motor 139 (via the controller 140) to rotate the drive wheel 138, thereby causing the curtain 130 to move along the path defined by the channels 136. The curtain 130 moves until the gap 134 is positioned above the vial 122i, after which the user can remove the vial 122i from the container 110.

The container 110 may take other configurations. For example, and referring to FIG. 10, a curtain 230 may be formed from multiple rigid panels 232 that are pivotally connected with each other, much like a conventional multi-panel garage door. In such an arrangement, each of the panels 232 may have pins or posts 237 received in channels in the side walls of the container 110 to guide the curtain 230 as it travels. The curtain 230 may be driven by a worm screw-traveler combination, wherein the traveler is fixed to one of the panels 232 located on the lower run of the curtain 230.

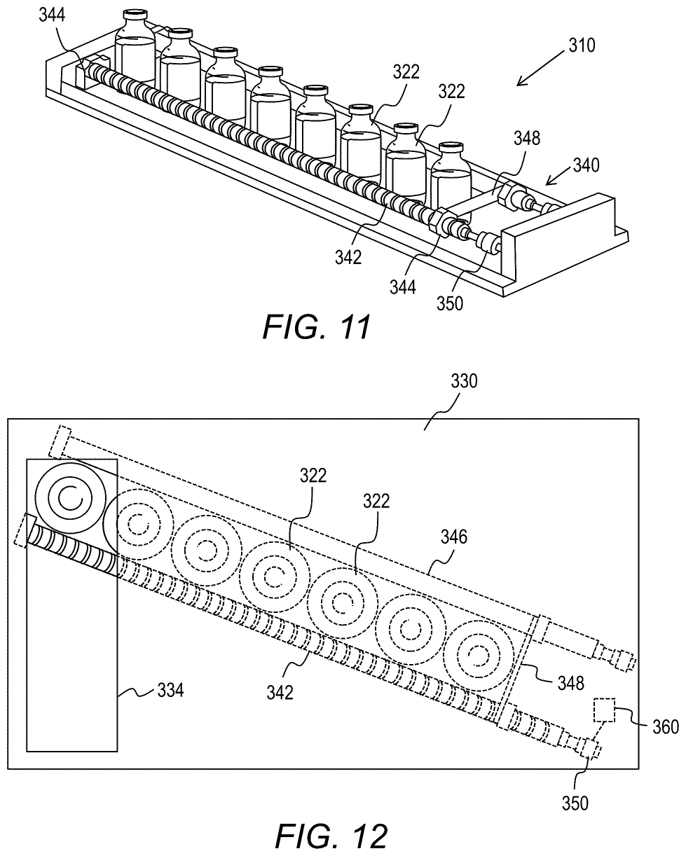

As another example, and as shown in FIGS. 11 and 12, a container 310 may lack a movable curtain, but may instead have a stationary curtain 330 that is open (i.e., has a gap 334) at one end. The container 310 also includes a track 320 that holds vials 322. Rather than the curtain 330 moving, the container 310 includes a drive unit 340 that is configured to move vials 322 one at a time in the track 320 to a position beneath the gap 334. The drive unit 340 has a drive screw 342 one on side of the track 320 that is rotatably mounted at each end with brackets 344, a guide rod 346 mounted at each end on the opposite side of the track 320, and a carriage 348 that spans the track 320 and is configured to slide on the guide rod 346 and threadedly engage the drive screw 342. A motor 350 is positioned to rotate the drive screw 342.

In operation, the user loads the track 320 with vials 322 and closes the lid to the container 310. When a vial 322 is to be dispensed, the user activates the cabinet 200 via the user interface 218, which causes the lid of the container 310 to open, thereby providing access to the vial 322 that is positioned directly below the gap 334. When the lid of the container 310 is closed, the controller 360 signals the motor 350 to rotate the drive screw 342. Rotation of the drive screw 342 causes the carriage 348 to move forwardly along the track 320 (guided by the guide rod 346), which action pushes all of the vials 322 forwardly in the track 320. Thus, when the lid next opens for dispensing of a vial 322, the “next” vial 322 is positioned beneath the gap 334 and can be accessed.

It will also be understood that, in some implementations, the containers 110, 310 may be standalone units rather than residing within a cabinet 200. In such an instance, access for loading and dispensing may be controlled by an internal or external security system.

As another example of a different configuration, in some embodiments a curtain like that shown at 130 may be employed, but rather than relying on interaction between the curtain 130 and the channel 136 to maintain the curtain in a desired position on a specific path, the curtain may be maintained in place by a series of rollers (e.g., positioned at the “corners” of the curtain) that facilitate movement of the curtain while maintaining its endless configuration. Other arrangements to induce the curtain to follow a prescribed path may also be employed.

The foregoing is illustrative of the present invention and is not to be construed as limiting thereof. Although exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention.

What is claimed is:

1. A container for a machine for dispensing vials containing a medicament, the container comprising:
a housing including a receptacle with an internal compartment and a lid, the receptacle including a track in the internal compartment, the track configured to receive a plurality of medicament vials;
a curtain connected with the housing, the curtain configured to form a gap above the track sized to permit access to a vial stored in the track;
a drive mechanism mounted to the housing; and
a controller operatively connected with the drive mechanism;
wherein the drive mechanism is configured to receive signals from the controller to selectively drive either the curtain or vials in the track so that a single vial is positioned below the gap.

2. The container defined in claim 1, wherein the lid is attached to the receptacle via a lid mechanism that is configured such that the cavity of the receptacle is inaccessible to a user unless the controller provides a signal indicating permission to access.

3. The container defined in claim 1, wherein the drive mechanism is connected with the curtain and is configured to drive the curtain relative to the housing.

4. The container defined in claim 1, wherein the drive mechanism is connected with the housing and configured to convey vials within the track.

5. The container defined in claim 1, wherein the curtain is configured to follow an endless path.

6. The container defined in claim 1, wherein the curtain comprises a unitary component and includes alternating flexible and rigid sections.

7. The container defined in claim 1, wherein the curtain comprises a plurality of pivotally interconnected panels.

8. The container defined in claim 1, wherein the housing and the controller are configured so that mounting of the container in an automated dispensing machine operatively connects the container with an operations controller of the automated dispensing machine, the operations controller configured to signal the controller of the container when permission to dispense a vial is granted.

9. The container defined in claim 1, wherein the lid is attached to the receptacle via a locking mechanism that is configured such that the cavity of the receptacle is inaccessible to a user unless the controller provides a signal indicating permission to access.

10. The container defined in claim 1, mounted in an automated dispensing machine.

11. A container for a machine for dispensing vials containing a medicament, the container comprising:
a housing including a receptacle with an internal compartment and a lid, the receptacle including a track in the internal compartment, the track configured to receive a plurality of medicament vials;
a curtain connected with the housing, the curtain configured to form a gap above the track sized to permit access to a vial stored in the track;
a drive mechanism mounted to the housing and to the curtain; and
a controller operatively connected with the drive mechanism;
wherein the drive mechanism is configured to receive signals from the controller to selectively drive the curtain relative to the housing so that a single vial is positioned below the gap.

12. The container defined in claim 11, wherein the lid is attached to the receptacle via a lid mechanism that is configured such that the cavity of the receptacle is inaccessible to a user unless the controller provides a signal indicating permission to access.

13. The container defined in claim 11, wherein the curtain comprises a unitary component and includes alternating flexible and rigid sections.

14. The container defined in claim 11, wherein the curtain comprises a plurality of pivotally interconnected panels.

15. The container defined in claim 11, wherein the housing and the controller are configured so that mounting of the container in an automated dispensing machine operatively connects the container with an operations controller of the automated dispensing machine, the operations controller configured to signal the controller of the container when permission to dispense a vial is granted.

16. The container defined in claim 11, wherein the lid is attached to the receptacle via a locking mechanism that is configured such that the cavity of the receptacle is inaccessible to a user unless the controller provides a signal indicating permission to access.

17. The container defined in claim 11, mounted in an automated dispensing machine.

18. A container for a machine for dispensing vials containing a medicament, the container comprising:
a housing including a receptacle with an internal compartment and a lid, the receptacle including a track in the internal compartment, the track configured to receive a plurality of medicament vials;
a curtain connected with the housing, the curtain configured to form a gap above the track sized to permit access to a vial stored in the track;
a drive mechanism mounted to the housing; and
a controller operatively connected with the drive mechanism;
wherein the drive mechanism is configured to receive signals from the controller to selectively drive vials in the track so that a single vial is positioned below the gap.

19. The container defined in claim 18, wherein the lid is attached to the receptacle via a lid mechanism that is configured such that the cavity of the receptacle is inaccessible to a user unless the controller provides a signal indicating permission to access.

20. The container defined in claim 18, wherein the housing and the controller are configured so that mounting of the container in an automated dispensing machine operatively connects the container with an operations controller of the automated dispensing machine, the operations controller configured to signal the controller of the container when permission to dispense a vial is granted.
